(19) United States
(12) Patent Application Publication
(10) Pub. No.: US 2026/0152780 A1
(43) Pub. Date: Jun. 4, 2026

(54) DRY ANALYTICAL ELEMENT FOR BILE ACID ANALYSIS AND METHOD OF MEASURING BILE ACID

(71) Applicant: FUJIFILM Corporation, Tokyo (JP)

(72) Inventor: Yoshihiko ABE, Ashigarakami-gun (JP)

(73) Assignee: FUJIFILM Corporation, Tokyo (JP)

(21) Appl. No.: 19/458,285

(22) Filed: Jan. 23, 2026

Related U.S. Application Data
(63) Continuation of application No. PCT/JP2024/026307, filed on Jul. 23, 2024.

(30) Foreign Application Priority Data
Jul. 24, 2023 (JP) 2023-119555

(51) Int. Cl. C12Q 1/32

(52) U.S. Cl. CPC C12Q 1/32; C12Y 101/0105

(57) ABSTRACT

An object of the present invention is to provide a dry analytical element for bile acid analysis which is simple and inexpensive and can be used as a quantitative reagent for an amount of bile acids without being limited to a sulfate-conjugated type, and a method of measuring a bile acid using the dry analytical element for bile acid analysis. According to the present invention, there is provided a dry analytical element for bile acid analysis, including, on a support, in the following order, at least one water-soluble polymer layer and at least one spreading layer, in which at least one of the water-soluble polymer layer or the spreading layer contains 3α-hydroxysteroid dehydrogenase, a thionicotinamide coenzyme, a reduced nicotinamide coenzyme, and a buffer.

### CROSS-REFERENCE TO RELATED APPLICATIONS

This application is a Continuation of PCT International Application No. PCT/JP2024/026307 filed on Jul. 23, 2024, which claims priority under 35 U.S.C § 119 (a) to Japanese Patent Application No. 2023-119555 filed on Jul. 24, 2023. Each of the above application(s) is hereby expressly incorporated by reference, in its entirety, into the present application.

### BACKGROUND OF THE INVENTION

### 1. Field of the Invention

The present invention relates to a dry analytical element for bile acid analysis and a method of measuring a bile acid, using 3α-hydroxysteroid dehydrogenase, a thionicotinamide coenzyme, and a reduced nicotinamide coenzyme.

### 2. Description of the Related Art

The bile acid is synthesized by an oxidation reaction of cholesterol in the liver. The synthesized bile acid is conjugated with taurine, glycine, sulfate, or glucuronic acid to form various structures. The bile acid thus synthesized is stored and concentrated in the gallbladder. The bile acid acts as a surfactant, and thus plays a role in promoting absorption of fat, bactericidal action, regulation of cholesterol metabolism, and the like.

The clinical significance of measuring the bile acid in screening for liver or biliary tract diseases is high, and generally, body fluids, for example, blood (whole blood, blood plasma, or serum), cerebrospinal fluid, lymph fluid, saliva, or urine are measured. However, since the bile acids are released into the blood due to a liver disease or a biliary tract disease, the measurement of the bile acid concentration in the blood is useful for liver function tests.

With respect to measurement of a bile acid by a measurement method in a solution (solution method), methods using enzymatic cycling are known, and JP1991-224498A (JP-H3-224498A) discloses a method of reacting 3a-hydroxysteroid dehydrogenase (hereinafter, also referred to as “3α-HSD”) with reduced nicotinamide coenzymes (hereinafter, also referred to as “NADHs”) and a thionicotinamide coenzyme (thio-NAD) (or thio-NADP) as coenzymes to measure the amount of generated thio-NADHs. On the other hand, JP1996-131193A (JP-H8-131193A) discloses a dry chemistry reagent for a sulfate-conjugated bile acid.

### SUMMARY OF THE INVENTION

In the measurement of the bile acid by the solution method disclosed in JP1991-224498A (JP-H3-224498A), to quantitatively determine the amount of the bile acid by the dye concentration, it is necessary to perform an operation such as calibration with a dedicated calibrator before the measurement, and a pipe facility for water supply and drainage and a large and expensive measuring device are required. Therefore, there is a demand for development of a simple and inexpensive measuring reagent for the bile acid.

However, the method described in JP1996-131193A (JP-H8-131193A) is limited to the “sulfate-conjugated type”, and it is considered that the “sulfate-conjugated bile acid” is synthesized in the liver and is mainly excreted in the urine. The measurement of the sulfate-conjugated bile acid is useful for an examination of a disease such as liver cirrhosis, but it has been pointed out that the proportion of the sulfate-conjugated bile acid present in the blood is several percent of the total bile acid in the blood, and the examination of the bile acid other than the sulfate-conjugated bile acid is important for examination of liver function such as acute hepatitis, chronic liver disease, or the intestinal absorption examination. Furthermore, the method described in JP1996-131193A (JP-H8-131193A) is promising for the qualitative measurement of the “sulfate-conjugated bile acid”, but since the linearity (straightness) of the signal concentration to be detected with respect to the amount of the “sulfate-conjugated bile acid” is insufficient, it is also difficult to use it as a quantitative reagent.

An object to be achieved by the present invention is to provide a dry analytical element for bile acid analysis which is simple and inexpensive and can be used as a quantitative reagent for an amount of bile acids without being limited to a sulfate-conjugated type, and a method of measuring a bile acid using the dry analytical element for bile acid analysis.

As a result of intensive studies to achieve the above object, the present inventors have found that a dry chemistry reagent that does not require a water supply and drainage facility and can be used as a quantitative reagent for an amount of bile acids can be provided, by a dry analytical element for bile acid analysis, which includes, on a support, in the following order, at least one water-soluble polymer layer and at least one spreading layer, in which at least one of the water-soluble polymer layer or the spreading layer contains 3α-hydroxysteroid dehydrogenase, a thionicotinamide coenzyme (thio-NAD), a reduced nicotinamide coenzyme (NADH), and a buffer. The present invention has been completed based on the above findings. According to the present invention, the following inventions are provided.
<1> A dry analytical element for bile acid analysis, comprising, on a support, in the following order: at least one water-soluble polymer layer; and at least one spreading layer, in which at least one of the water-soluble polymer layer or the spreading layer contains 3α-hydroxysteroid dehydrogenase, a thionicotinamide coenzyme, a reduced nicotinamide coenzyme, and a buffer.
<2> The dry analytical element for bile acid analysis according to <1>, in which the water-soluble polymer layer is a gelatin layer.
<3> The dry analytical element for bile acid analysis according to <1> or <2>, in which the buffer is a buffer having a buffering capacity in a pH range of 6.0 to 10.0.
<4> The dry analytical element for bile acid analysis according to any one of <1> to <3>, in which the buffer is tris(hydroxymethyl)aminomethane, 4-(2-hydroxyethyl)-1-piperazineethanesulfonic acid, N-[tris(hydroxymethyl)methyl]glycine, or glycine.
<5> The dry analytical element for bile acid analysis according to any one of <1> to <4>, in which the thionicotinamide coenzyme is thio-NAD, and the reduced nicotinamide coenzyme is NADH.
<6> The dry analytical element for bile acid analysis according to any one of <1> to <5>, in which a content of the thionicotinamide coenzyme is 0.05 to 0.6 g/m².
<7> The dry analytical element for bile acid analysis according to any one of <1> to <6>, in which a content of the reduced nicotinamide coenzyme is 0.05 to 0.6 g/m².
<8> The dry analytical element for bile acid analysis according to any one of <1> to <7>, in which a content of the 3α-hydroxysteroid dehydrogenase is 3.0 to 12.0 KU/m².
<9> A method of measuring a bile acid, comprising: spotting a bile acid-containing sample on the dry analytical element for bile acid analysis according to any one of <1> to <8>; and measuring color development.

According to the dry analytical element for bile acid analysis and the method of measuring a bile acid according to the present invention, it is possible to provide a dry chemistry reagent that does not require a water supply and drainage facility and can be used as a quantitative reagent for an amount of bile acids.

### BRIEF DESCRIPTION OF THE DRAWINGS

FIG. 1 shows a relationship between a ΔOD/min value and a bile acid concentration in Example 1.

FIG. 2 shows a relationship between a ΔOD/min value and a bile acid concentration in Example 2.

FIG. 3 shows a relationship between a ΔOD/min value and a bile acid concentration in Example 3.

FIG. 4 shows a relationship between a ΔOD/min value and a bile acid concentration in Example 4.

FIG. 5 shows a relationship between a ΔOD/min value and a bile acid concentration in Example 5.

FIG. 6 shows a relationship between a ΔOD/min value and a bile acid concentration in Example 6.

### DESCRIPTION OF THE PREFERRED EMBODIMENTS

Hereinafter, embodiments of the present invention will be specifically described. In the present specification, a numerical range indicated using “to” represents a range including numerical values described before and after “to” as a minimum value and a maximum value.

The present invention relates to a dry analytical element for bile acid analysis, comprising, on a support, in the following order: at least one water-soluble polymer layer; and at least one spreading layer, in which at least one of the water-soluble polymer layer or the spreading layer contains 3α-hydroxysteroid dehydrogenase, a thionicotinamide coenzyme, a reduced nicotinamide coenzyme, and a buffer.

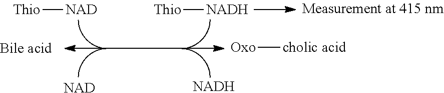

The method according to the embodiment of the present invention uses a reaction system in which 3α-hydroxysteroid dehydrogenase (3α-HSD) acts as a catalyst. As shown above, in a case where the bile acid is present, the thionicotinamide coenzyme (thio-NAD) and the reduced nicotinamide coenzyme (NADH) are changed to the reduced thionicotinamide coenzyme (thio-NADH) and the nicotinamide coenzyme (NAD). In the above-described reaction system, since the color development of thio-NADH is correlated with the reaction amount of the bile acid, the amount of the bile acid can be quantified by measuring the color development of thio-NADH. Therefore, in a case where the amount of 3α-hydroxysteroid dehydrogenase (3α-HSD) is adjusted, the reaction rate of the amount of the analyte fluctuates depending on the amount of the bile acid, which is the analyte, so that a difference occurs in the rate method or in the color development amount (OD) at a fixed time. In this way, the bile acid can be quantified.

The water-soluble polymer layer used in the present invention represents a layer containing a water-soluble polymer provided on a support. The water-soluble polymer layer plays a role of spreading a specimen containing a bile acid spotted on the spreading layer described below substantially uniformly in the analysis element by drawing in the specimen into the water-soluble polymer layer. As a preferred water-soluble polymer contained in the water-soluble polymer layer, gelatin is preferable. That is, the water-soluble polymer layer is preferably a gelatin layer. The water-soluble polymer has a particularly swelling property. A usage amount of the water-soluble polymer is not particularly limited, but is preferably 8.0 g/m² or more and 40.0 g/m² or less, and more preferably 15.0 g/m² or more and 30.0 g/m² or less.

The spreading layer used in the present invention is a layer having an action (metering action) of spreading an aqueous liquid sample spotted and supplied to an upper surface of the dry analytical element for bile acid analysis in a lateral direction without substantially distributing a component contained in the aqueous liquid sample unevenly, and supplying the aqueous liquid sample to a lower layer containing an absorbent water-soluble polymer in a substantially constant proportion per unit area.

As the spread layer used in the present invention, for example, a woven spreading layer (for example, a plain woven fabric such as broadcloth or poplin) described in JP1980-164356A (JP-S55-164356A), JP1982-66359A (JP-S57-66359A), or the like; a knitted spreading layer (for example, a tricot knitted fabric, a double tricot knitted fabric, a Milanese knitted fabric, or the like) described in JP1985-222769A (JP-S60-222769A) or the like; a spreading layer consisting of wet-laid paper containing organic polymer fiber pulp as described in JP1982-148250A (JP-S57-148250A); a nonfibrous isotropic porous spreading layer such as a membrane filter (brush polymer layer) as described in JP1978-21677A (JP-S53-21677A) and U.S. Pat. No. 3,992,158A, or a continuous microporous porous layer in which polymer microbeads, glass microbeads, or diatomaceous earth are held by a water-soluble polymer binder; a nonfibrous isotropic porous spreading layer composed of a continuous microporous porous layer (three-dimensional lattice-like particulate structure layer) in which polymer microbeads are bond in point contact with each other by a polymer adhesive that does not swell or expand in water as described in JP1980-90859A (JP-S55-90859A); or the like can be used. As the spreading layer, a knitted spreading layer (for example, a tricot knitted fabric, a double tricot knitted fabric, a Milanese knitted fabric, or the like) is preferable.

In addition, an interlayer such as an adhesive layer can also be provided between the support and the layer provided thereon and between the respective layers provided on the support.

As the support, a water-impermeable support is preferable. As the material of the water-impermeable support, a polymer such as polyethylene terephthalate, polycarbonate of bisphenol A, polystyrene, or cellulose ester (for example, cellulose diacetate, cellulose triacetate, cellulose acetate propionate, or the like) is preferable, and polyethylene terephthalate is particularly preferable. As the support, a smooth planar support that is transparent, for example, transmits electromagnetic radiation having a wavelength in a range of at least a part of a wavelength range of approximately 200 nm to approximately 900 nm, in a thickness range of approximately 50 μm to approximately 1 mm and preferably approximately 80 μm to approximately 300 μm can be used. A known undercoat or adhesive layer can be provided on the surface of the support to strengthen the adhesion to the interlayer.

In the dry analytical element for bile acid analysis according to the embodiment of the present invention, at least one of the water-soluble polymer layer or the spreading layer contains 3α-hydroxysteroid dehydrogenase (3α-HSD). The 3α-hydroxysteroid dehydrogenase is an enzyme that oxidizes 3α-hydroxysteroid, and is a conjugated enzyme. As the content of the 3α-hydroxysteroid dehydrogenase (3α-HSD), to ensure sensitivity, 3.0 to 12.0 KU/m² is preferable and 4.0 to 8.0 KU/m² is more preferable.

The dry analytical element for bile acid analysis according to the embodiment of the present invention contains an (oxidized) thionicotinamide coenzyme. The (oxidized) thionicotinamide coenzyme is a coenzyme that functions by binding to various oxidoreductases similarly to the nicotinamide coenzyme, and is involved in biological hydrogen transfer, and specifically means thio-NAD or thio-NADP. As the content of the (oxidized) thionicotinamide coenzyme, to ensure sensitivity, 0.05 to 1.0 g/m² is preferable, 0.05 to 0.6 g/m² is more preferable, and 0.1 to 0.5 g/m² is still more preferable. It is preferable that the content of the (oxidized) thionicotinamide coenzyme is 0.6 g/m² or less because the background decreases.

The dry analytical element for bile acid analysis according to the embodiment of the present invention contains reduced nicotinamide coenzyme. The reduced nicotinamide coenzyme is a coenzyme of various dehydrogenases, and specifically means NADH or NADPH. As the usage amount of the reduced nicotinamide coenzyme, to ensure sensitivity, 0.05 to 1.0 g/m² is preferable, 0.05 to 0.6 g/m² is more preferable, and 0.1 to 0.5 g/m² is still more preferable. It is preferable that the content of the reduced nicotinamide coenzyme is 0.6 g/m² or less because the background decreases.

The dry analytical element for bile acid analysis according to the embodiment of the present invention contains a buffer.

The buffer is preferably a buffer having a buffering capacity in a pH range of 6.0 to 10.0, and more preferably a buffer having a buffering capacity in a pH range of 7.0 to 9.0.

Examples of the type of the buffer include known buffers such as tris(hydroxymethyl)aminomethane, glycine, a phosphate, and a Good's buffer. The buffer is preferably tris(hydroxymethyl)aminomethane (also referred to as Tris), 4-(2-hydroxyethyl)-1-piperazineethanesulfonic acid (also referred to as HEPES), N-[tris(hydroxymethyl)methyl]glycine (also referred to as Tricine), N,N-bis(2-hydroxyethyl)glycine (also referred to as Bicine), or glycine. The buffer is more preferably tris(hydroxymethyl)aminomethane, 4-(2-hydroxyethyl)-1-piperazineethanesulfonic acid, N-[tris(hydroxymethyl)methyl]glycine, or glycine.

A content of the buffer is not particularly limited as long as it does not affect the pH of the specimen, but is preferably 1.0 to 10.0 g/m² and more preferably 3.0 to 8.0 g/m².

In the present invention, the amount of the bile acid can be quantified by containing 3α-hydroxysteroid dehydrogenase (3α-HSD), a thionicotinamide coenzyme, a reduced nicotinamide coenzyme, and a buffer in at least one of the water-soluble polymer layer or the spreading layer. That is, the 3α-hydroxysteroid dehydrogenase, the (oxidized) thionicotinamide coenzyme, the reduced nicotinamide coenzyme, and the buffer may be contained in the water-soluble polymer layer, may be contained in the spreading layer, or may be contained in both the water-soluble polymer layer and the spreading layer. It is more preferable that these are contained in the spreading layer.

The dry analytical element for bile acid analysis according to the embodiment of the present invention may be an analytical element further including a reagent layer, a reflective layer, a light shielding layer, a filtering layer, an undercoat layer, and other layers. Examples of such an analytical element include those disclosed in U.S. Pat. Nos. 3,992,158A and 4,042,335A, but as a preferred configuration of the present invention, an integral multi-layer analytical element produced by sequentially laminating and integrating a water-soluble polymer layer having a water-absorbing function and a spreading layer that spreads a specimen in a lateral direction on a support having an optical transmittance and water-impermeability is preferable.

The dry analytical element for bile acid analysis according to the embodiment of the present invention can be prepared by a method known to those skilled in the art. For example, a coating liquid prepared as an interlayer coating liquid is applied onto the support and dried to produce a dried film having a thickness of about 40 μm, and then a woven fabric of the spreading layer is bonded thereto. Thereafter, a coating liquid blended as a reagent holding layer liquid is applied onto the spreading layer from the woven fabric side and dried to produce a dry analytical element for bile acid analysis. From various viewpoints such as manufacturing, packaging, transportation, storage, and measurement operation, it is preferable that the dry analytical element for bile acid analysis is cut into a small piece which is a square having one side of about 15 mm to about 30 mm, a substantially same size and shape, or the like, and is stored in a slide frame described in JP1982-28331B (JP-S57-28331B), JP1981-142454U (JP-S56-142454U), JP1982-63452A (JP-S57-63452A), JP1983-32350U (JP-S58-32350U), JP1983-501144A (JP-S58-501144A), and the like to be used as a chemical analysis slide. Depending on the intended use, the dry analytical element for bile acid analysis can be used in a cassette or a magazine in a long tape shape, or can be used by being attached to or stored in a card having an opening in a small piece.

According to the present invention, there is provided a method of measuring a bile acid, the method including: spotting a bile acid-containing sample on the dry analytical element for bile acid analysis according to the embodiment of the present invention; and measuring color development.

For example, the content of bile acid in the liquid sample can be determined by spotting an aqueous liquid sample such as whole blood, blood plasma, serum, lymph fluid, or urine, in a range of approximately 5 μL to approximately 30 μL and preferably approximately 8 μL to approximately 15 μL, on the spreading layer, incubating the aqueous liquid sample at a substantially constant temperature in a range of approximately 20° C. to approximately 40° C. and preferably at a substantially constant temperature in the vicinity of 37° C. for a range of approximately 1 minute to approximately 10 minutes and preferably approximately 2 minutes to approximately 7 minutes, and measured, by reflection photometry, the detectable change such as the color change or the color development in the dry analytical element for bile acid analysis from the support side, according to the principle of the colorimetry. In the present invention, the optical density of the spreading layer is measured by reflection photometry using light having an absorption maximum wavelength of a bile acid or a wavelength in the vicinity thereof, and the bile acid content in the liquid sample can be determined according to the principle of the colorimetry using a calibration curve created in advance. By keeping the amount of the aqueous liquid sample to be spotted, the incubation time, and the incubation temperature constant, the quantitative analysis of the bile acid can be performed with high accuracy. In the measurement operation, it is possible to perform a highly accurate quantitative analysis by a chemical analysis apparatus described in JP1985-125543A (JP-S60-125543A), JP1985-220862A (JP-S60-220862A), JP1986-294367A (JP-S61-294367A), JP1983-161867A (JP-S58-161867A), and the like in an extremely easy operation.

Next, the present invention will be described using Examples, but the present invention is not limited thereto.

### EXAMPLES

### Example 1

### (1) Production of Coating Film and Dried Slide

A water solution having the following composition-1 was applied onto a smooth colorless transparent polyethylene terephthalate (PET) film having a thickness of 180 μm and having a gelatin subbing layer, such that the thickness after drying was 40 μm, and dried to provide a water-absorbing layer.

### Water-Absorbing Layer (Composition-1)

Gelatin, 17 g/m²
Surfactant, 0.2 g/m²
Here, polyoxy (2-hydroxy) propylene nonylphenyl ether (Surfactant 10G, manufactured by Olin Corporation) was used as the surfactant.

Next, water was supplied to the front surface of the above-described film at a supply amount of about 30 g/m² to be moistened, and then a polyester spun yarn tricot knitted fabric having a denier of 50 was bonded thereto using a wet lamination method while applying a slight pressure to provide a spreading layer.

Next, a water solution A having the following composition was applied onto the spreading layer such that each component was in the following amount, and dried to produce a dry analytical element for bile acid analysis according to the present invention.

### Aqueous Solution A

3α-hydroxysteroid dehydrogenase (3α-HSD) (manufactured by TOYOBO Co., Ltd.), 6.4 KU/m²
Thio-NAD (manufactured by Oriental Yeast Co., Ltd.), 0.32 g/m²
NADH (manufactured by Oriental Yeast Co., Ltd.), 0.32 g/m²
Tris(hydroxymethyl)aminomethane (manufactured by FUJIFILM Wako Pure Chemical Corporation), 8.7 g/m²
Polyvinylpyrrolidone (manufactured by BASF SE), 10.9 g/m²

The above-described dry analytical element for bile acid analysis was cut into a size of 12 mm×13 mm, a slide was prepared according to the method described in JP1982-63452A (JP-S57-63452A), and a dried analysis slide (1) for bile acid analysis was produced.

### (2) Measurement of Bile Acid Concentration

A pooled human serum specimen prepared such that the bile acid concentration was 48.9 μmol/L, 117.8 μmol/L, or 185.8 μmol/L, and a 7% human serum albumin (HSA) aqueous solution as a measurement reagent having a bile acid concentration of zero were prepared, 10 μL of each was spotted on the dried analysis slide (1) prepared in Example 1, and the reflection concentration at 415 nm was measured by a FUJIFILM Dry Chem 7000 analyzer (manufactured by FUJIFILM Corporation) every 10 seconds for 3 minutes while maintaining the temperature at 37° C.

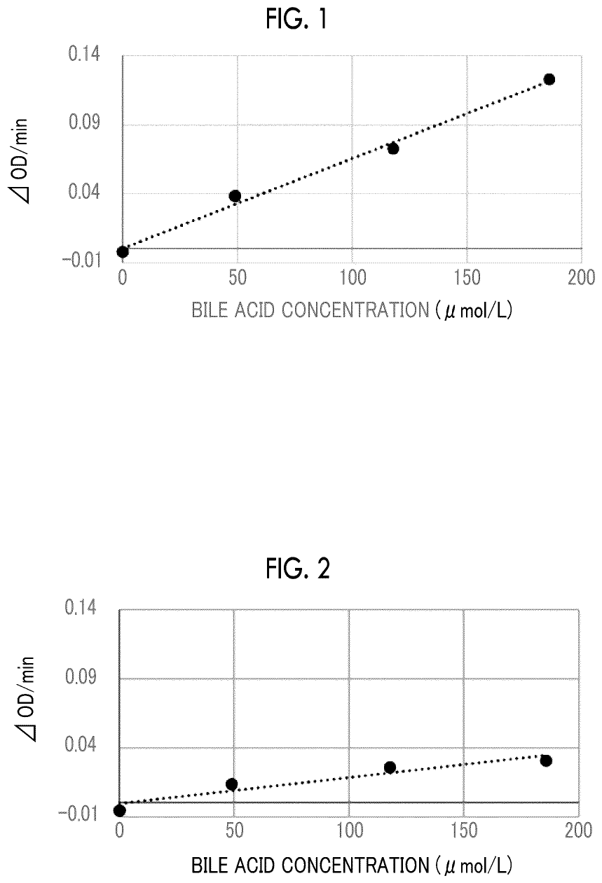

The concentration of the bile acid and the amount of change in the reflection concentration per minute (ΔOD/min) in the reflection concentration that increases between 60 sec and 180 sec of the measurement time were summarized in Table 1, and a relationship of the ΔOD/min value to the concentration of the bile acid was shown in FIG. 1, in which the concentration of the bile acid was taken as the horizontal axis and the ΔOD value was taken as the vertical axis.

TABLE 1

| Bile acid (μmol/L) | ΔOD/min |
|---|---|
| 0 | −0.0023 |
| 48.9 | 0.038 |
| 117.8 | 0.0724 |
| 185.8 | 0.1227 |

### Example 2

### (1) Production of Coating Film and Dried Slide

An aqueous solution having the following composition-2 was applied onto a smooth colorless transparent PET film having a thickness of 180 μm and having a gelatin subbing layer, such that the thickness after drying was 40 μm, and dried to provide a water-absorbing layer.

### Water-Absorbing Layer (Composition-2)

Polyvinyl alcohol, 23 g/m²
Surfactant, 0.2 g/m²
Here, polyoxy (2-hydroxy) propylene nonylphenyl ether (Surfactant 10G, manufactured by Olin Corporation) was used as the surfactant.

Next, a polyester spun yarn tricot knitted fabric having a denier of about 50 was bonded thereto using a wet lamination method to provide a spreading layer in the same manner as in Example 1.

In addition, the water solution A described in Example 1 was applied onto the spreading layer such that the amount was the same as in Example 1, and dried to produce a dried analysis slide (2) for bile acid analysis according to the present invention.

### (2) Measurement of Bile Acid Concentration

A pooled human serum specimen prepared such that the bile acid concentration was 48.9 μmol/L, 117.8 μmol/L, or 185.8 μmol/L, and a 7% HSA aqueous solution as a measurement reagent having a bile acid concentration of zero were prepared, 10 μL of each was spotted on the dried analysis slide (2) prepared in Example 2, and the reflection concentration at 415 nm was measured by a FUJIFILM Dry Chem 7000 analyzer (manufactured by FUJIFILM Corporation) every 10 seconds for 3 minutes while maintaining the temperature at 37° C.

The concentration of the bile acid and the amount of change in the reflection concentration per minute (ΔOD/min) in the reflection concentration that increases between 60 sec and 180 sec of the measurement time were summarized in Table 2, and a relationship of the ΔOD/min value to the concentration of the bile acid was shown in FIG. 2, in which the concentration of the bile acid was taken as the horizontal axis and the ΔOD value was taken as the vertical axis.

TABLE 2

| Bile acid (μmol/L) | ΔOD/min |
|---|---|
| 0 | −0.0059 |
| 48.9 | 0.0135 |
| 117.8 | 0.0256 |
| 185.8 | 0.0304 |

From the results of Examples 1 and 2, it can be seen that both the dry analytical elements for bile acid analysis have favorable linearity and exhibit sufficient performance as a reagent capable of quantitatively measuring the bile acid. It is more preferable to use gelatin as the water-absorbing layer in the dry analytical element because a larger signal-to-noise ratio (S/N) can be obtained.

### Examples 3 to 6

The following level was prepared as a buffer, and the slide was confirmed.

An experiment (Example 3) was repeated using the aqueous solution A of Example 1, and the Tris(hydroxymethyl)aminomethane (Tris) in Example 3 was changed to HEPES (Example 4), Tricine (Example 5), or Glycine (Example 6), and a dried analysis slide was produced in the same manner as in Example 1. In the same manner as in Example 1, a pooled human serum specimen was prepared such that the bile acid concentration (solution method measurement value) was 0 μmol/L, 118 μmol/L, or 186 μmol/L, 10 μL of each was spotted on the dried analysis slide using the above-described buffer solution, and the reflection concentration at 415 nm was measured by a FUJIFILM Dry Chem 7000 analyzer (manufactured by FUJIFILM Corporation) every 10 seconds for 3 minutes while maintaining the temperature at 37° C. The number of specimens was N=2.

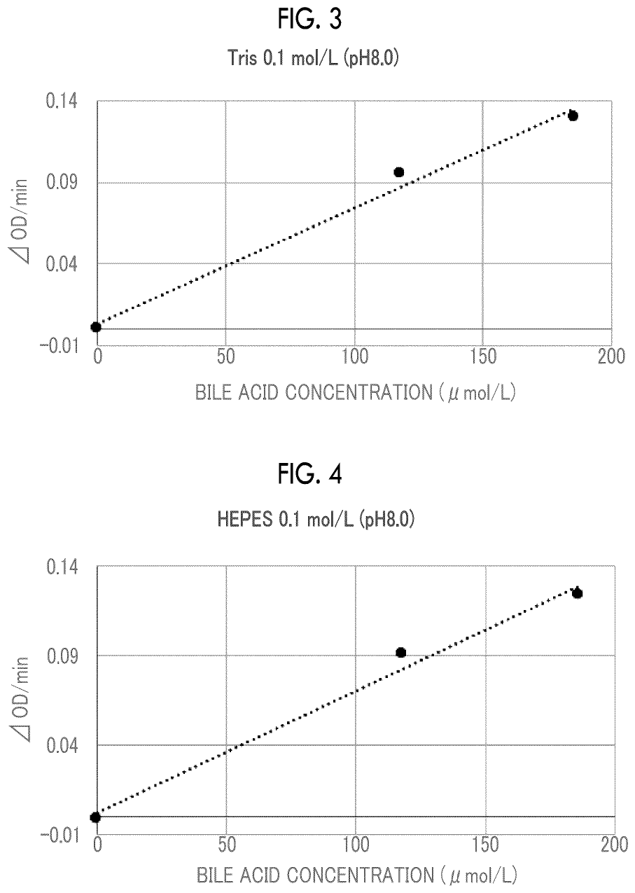

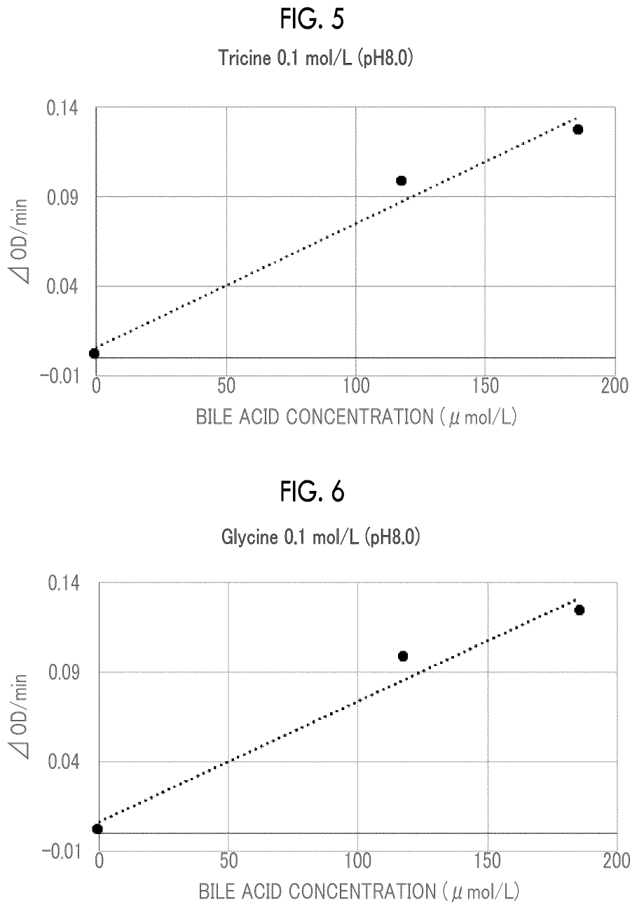

The concentration of the bile acid and the amount of change in the reflection concentration per minute (ΔOD/min) in the reflection concentration that increases between 60 sec and 180 sec of the measurement time were summarized in Table 3, and a relationship of the ΔOD/min value to the concentration of the bile acid was shown in FIGS. 3 to 6, in which the concentration of the bile acid was taken as the horizontal axis and the ΔOD value was taken as the vertical axis.

TABLE 3

| Bile acid | Tris | HEPES | Tricine | Glycine |
|---|---|---|---|---|
| (μmol/L) | ΔOD/min | ΔOD/min | ΔOD/min | ΔOD/min |
| 0 | 0.001 | 0.000 | 0.002 | 0.002 |
| 118 | 0.095 | 0.091 | 0.099 | 0.098 |
| 186 | 0.130 | 0.124 | 0.127 | 0.124 |

From the results of Examples 3 to 6, it can be seen that both the dry analytical elements for bile acid analysis have favorable linearity and exhibit sufficient performance as a reagent capable of quantitatively measuring the bile acid.

What is claimed is:

1. A dry analytical element for bile acid analysis, comprising, on a support, in the following order:
at least one water-soluble polymer layer; and
at least one spreading layer,
wherein at least one of the water-soluble polymer layer or the spreading layer contains 3α-hydroxysteroid dehydrogenase, a thionicotinamide coenzyme, a reduced nicotinamide coenzyme, and a buffer.

2. The dry analytical element for bile acid analysis according to claim 1,
wherein the water-soluble polymer layer is a gelatin layer.

3. The dry analytical element for bile acid analysis according to claim 1,
wherein the buffer is a buffer having a buffering capacity in a pH range of 6.0 to 10.0.

4. The dry analytical element for bile acid analysis according to claim 1,
wherein the buffer is tris(hydroxymethyl)aminomethane, 4-(2-hydroxyethyl)-1-piperazineethanesulfonic acid, N-[tris(hydroxymethyl)methyl]glycine, or glycine.

5. The dry analytical element for bile acid analysis according to claim 1,
wherein the thionicotinamide coenzyme is thio-NAD, and the reduced nicotinamide coenzyme is NADH.

6. The dry analytical element for bile acid analysis according to claim 1,
wherein a content of the thionicotinamide coenzyme is 0.05 to 0.6 g/m².

7. The dry analytical element for bile acid analysis according to claim 1,
wherein a content of the reduced nicotinamide coenzyme is 0.05 to 0.6 g/m².

8. The dry analytical element for bile acid analysis according to claim 1,
wherein a content of the 3α-hydroxysteroid dehydrogenase is 3.0 to 12.0 KU/m².

9. A method of measuring a bile acid, comprising:
spotting a bile acid-containing sample on the dry analytical element for bile acid analysis according to claim 1; and
measuring color development.
